(19) United States
(12) Patent Application Publication
(10) Pub. No.: US 2008/0051691 A1
(43) Pub. Date: Feb. 28, 2008

(54) Implantable shunt or catheter enabling gradual delivery of therapeutic agents

(75) Inventors: Deborah L. Dragoon, Altona, NY (US); Jonathan Marc Cohen, Monroe, NY (US)

Correspondence Address: HOWSON AND HOWSON/WYETH;CATHY A. KODROFF, SUITE 210, 501 OFFICE CENTER DRIVE, FT WASHINGTON, PA, 19034, US

(73) Assignee: Wyeth, Madison, NJ (US)

(21) Appl. No.: 11/895,770

(22) Filed: Aug. 27, 2007

Related U.S. Application Data
(60) Provisional application No. 60/840,591, filed on Aug. 28, 2006.

(51) Int. Cl. A61M 27/00; A61K 31/436; A61P 25/00; A61K 9/00

(52) U.S. Cl. 604/8; 424/423; 514/291

(57) ABSTRACT

An implantable catheter or shunt for draining fluid from a body cavity. The catheter or shunt body has a wall structure that carries one or more therapeutic agents in a manner enabling release of the therapeutic agent from the wall structure in situ after surgical implantation of the catheter or shunt body. The therapeutic agent can be gradually released over time to prevent infection, inhibit tissue ingrowths, and/or provide some other desired medicinal purpose. As an example, the therapeutic agent can be rapamycin or an mTOR inhibitor. According to some contemplated embodiments of the present invention, the therapeutic agent carried by the catheter/shunt is rechargeable or refillable in situ so that the therapeutic agent can be gradually released from the catheter/shunt over the expected useful life of the catheter/shunt.

### CROSS-REFERENCE TO RELATED APPLICATIONS

This application claims the benefit under 35 USC 119(e) of U.S. Provisional Patent Application No. 60/840,591, filed Aug. 28, 2006.

### BACKGROUND OF THE INVENTION

The present invention relates, in general, to surgical devices such as implantable catheters and shunts for draining fluids between different areas within the human body, and more particularly, to catheters and shunts that minimize risks of blockage and obstruction and/or infection.

Shunts and catheters have been employed in surgical applications for controlling the flow of body fluids between various regions of a human body. As one example, implantable shunt systems are used in the treatment of hydrocephalus to overcome or control the lack of free circulation and/or absorption of cerebrospinal fluid within the human brain.

Hydrocephalus is a neurological condition that is caused by the abnormal accumulation of cerebrospinal fluid within the ventricles, or cavities, of the brain. The cerebrospinal fluid surrounds the brain and spinal cord and circulates through the ventricular system of the brain to provide a protective cushion for the brain and spinal cord. Hydrocephalus arises when normal drainage of cerebrospinal fluid is blocked creating an imbalance between the amount of the fluid being produced by the choroid plexus and the rate at which the fluid is absorbed into the bloodstream. Such an imbalance increases pressure on the brain and causes the ventricles to enlarge.

Treating hydrocephalus typically involves the surgical placement of cerebrospinal fluid shunts, which provide a mechanical system of valves and tubes that divert a controlled amount of the fluid out of the cranial cavity and into another region of the body where the fluid can be absorbed. The proximal end of a ventricular catheter is placed within the ventricles to provide a drainage path leading out of the brain to a valved drainage shunt that directs the fluid, for instance, to the abdomino-peritoneal cavity where the cerebrospinal fluid can be absorbed in peritoneal fluid and into the bloodstream.

A problem experienced with the implantation of such catheters and shunts is that the inflow end of a ventricular catheter can become obstructed or blocked due to ingrowths of choroid tissue. This renders the system inoperative in relieving excess pressure and requires surgery to remove the system without tearing the brain tissue or causing bleeding.

Another problem is that of infection. As a foreign object to the body, the implanted catheter or shunt provides a suitable site for microorganism growth. Infection commonly becomes evident within about seven to ninety days after implantation. In the event of an infection, the shunt system is typically removed.

Examples of cerebrospinal fluid shunt systems are described in U.S. Pat. No. 7,037,288 B2 issued to Rosenberg et al.; U.S. Pat. No. 5,531,673 issued to Helenowski; U.S. Pat. No. 5,405,316 issued to Magram; U.S. Pat. No. 4,950,232 issued to Ruzicka et al.; 4,655,745 issued to Corbett; and U.S. Pat. No. 4,382,445 issued to Sommers.

### SUMMARY OF THE INVENTION

The present invention relates to an implantable catheter or shunt system for draining fluid from a body cavity. The catheter or shunt body has a wall structure that carries one or more therapeutic agents in a manner enabling slow delivery of the therapeutic agent or agents from the wall structure in situ after surgical implantation of the catheter or shunt body. As an example, release of the therapeutic agent or agents can be provided for the purposes of preventing infection, inhibiting tissue ingrowths, and/or performing some other desired medicinal function. In some contemplated embodiments of the present invention, the supply of the therapeutic agent carried by the wall structure of the implanted catheter/shunt is rechargeable and/or refillable in situ so that the gradual release of the therapeutic agent can be accomplished over an extended period of time such as the intended useful lifespan of the implanted catheter/shunt.

The invention provides an implantable shunt system for draining fluid from a body cavity, comprising a catheter and at least one therapeutic agent carried by a wall structure of said catheter, said wall structure having an outer peripheral surface and an inner peripheral surface defining a lumen through which fluid is drained from the body cavity, and said wall structure enabling gradual release of said therapeutic agent from said wall structure in situ after implantation of said catheter body.

The invention provides an implantable shunt system capable of draining fluid from a body cavity, comprising a catheter and at least one therapeutic agent carried by a wall structure of said catheter, said wall structure having an outer peripheral surface and an inner peripheral surface defining a lumen through which fluid is to be drained from the body cavity, and said wall structure capable of enabling gradual release of said therapeutic agent from said wall structure in situ after implantation of said catheter body.

The invention provides an implantable shunt system adapted to drain fluid from a body cavity, comprising a catheter and at least one therapeutic agent carried by a wall structure of said catheter, said wall structure having an outer peripheral surface and an inner peripheral surface defining a lumen through which fluid is to be drained from the body cavity, and said wall structure adapted to enable gradual release of said therapeutic agent from said wall structure in situ after implantation of said catheter body.

The implantable shunt system preferably further comprising a means for recharging or refilling the therapeutic agent carried by the wall structure in situ within a patient.

The implantable shunt system may further comprise at least one channel extending within the wall structure between the inner and outer peripheral surfaces, the channel containing a supply of the therapeutic agent which is gradually releasable therefrom through at least one of the inner and outer peripheral surfaces. The implantable shunt system may further comprise at least one channel extending within the wall structure between the inner and outer peripheral surfaces, the channel being adapted to contain a supply of the therapeutic agent which can be gradually releasable from the channel through at least one of the inner and outer peripheral surfaces. The implantable shunt system may further comprise at least one channel extending within the wall structure between the inner and outer peripheral surfaces, the channel being adapted to contain a supply of the therapeutic agent, it is suitably adapted to gradually release the agent through at least one of the inner and outer peripheral surfaces.

At least one channel suitably includes an inlet. The catheter may further comprise a reservoir, to carry a supply of therapeutic agent, located external of the wall structure and in fluid communication with the inlet. The implantable shunt system may suitably further comprise a pump that can be actuated in order to pump the therapeutic agent from the reservoir into the channel via the inlet.

The implantable shunt system may suitably comprise a plurality of adjacent channels extending in a substantially longitudinal direction within the wall structure along a predetermined length of the catheter body. The end of at least one of the channels may interconnect with the end an adjacent channel so that the therapeutic agent may travel in a first direction along a length of one of the channels and in a reverse direction within the adjacent channel. The reservoir may include multiple separate reservoirs. These may each communicate with a different channel. They may each carry a supply of a different therapeutic agent. The implantable shunt system may be adapted to carry multiple different therapeutic agents are in the wall structure. The therapeutic agent may be provided in a film formed on a surface of the wall structure. The therapeutic agent may be impregnated within a material from which the wall structure is formed and be diffusible through the wall structure. The wall structure may be a single-walled tube extruded with hollow channels extending within the single-wall of the tube. The wall structure may include an inner tube defining the lumen through which fluid is to be drained from the body cavity and an outer tube or jacket supported a spaced distance about the inner tube defining a chamber therebetween suitable for containing a supply of the therapeutic agent. The wall structure may include at least one stabilizer positioned within the chamber to maintain proper spacing between the inner tube and outer tube or jacket or to divide the chamber into multiple separate chambers.

The invention provides a cerebrospinal fluid shunt system having a ventricular catheter and drainage shunt interconnected directly or indirectly by a flow control mechanism, one of the ventricular catheter and drainage shunt comprising an elongate body defining a lumen therein for passage of cerebrospinal fluid to or from the flow control mechanism, the body formed by a wall structure carrying at least one therapeutic agent therein or thereon, the wall structure releasing the therapeutic agent in situ after implantation of the shunt system.

The invention provides a cerebrospinal fluid shunt system comprising a ventricular catheter and drainage shunt interconnected directly or indirectly by a flow control mechanism, wherein one of the ventricular catheter and drainage shunt comprises an elongate body defining a lumen therein suitable for the passage of cerebrospinal fluid to or from the flow control mechanism in use and the body is formed by a wall structure carrying at least one therapeutic agent therein or thereon. The wall structure is capable of releasing the therapeutic agent in situ after implantation of the shunt system.

The cerebrospinal fluid shunt system may further comprise a means for recharging or refilling the therapeutic agent carried by the wall structure in situ within a patient. The therapeutic agent is suitable provided in a film formed on a surface of the wall structure or may be impregnated within a material from which the wall structure is composed. It is preferably diffusible through the wall structure.

The wall structure may provide an outer peripheral surface of the body and an inner peripheral surface of the body, wherein the inner peripheral surface defines the lumen, and preferably wherein at least one channel extends within the wall structure between the inner and outer peripheral surfaces, the channel preferably containing a supply of the therapeutic agent which is to be slowly releasable therefrom in use through at least one of the inner and outer peripheral surfaces. At least one channel may include an inlet and an outlet permitting flushing, refilling, recharging or circulating of the supply of therapeutic agent in the channel. The cerebrospinal fluid shunt system may further comprise at least one implantable reservoir carrying a supply of the therapeutic agent, located external of the wall structure, and in fluid communication with the inlet and outlet.

The cerebrospinal fluid shunt system may further comprise an implantable pump located external of the wall structure and adapted to pump the therapeutic agent from the reservoir into the channel. The wall structure may suitably be an extruded flexible single-walled tube having an array of separate longitudinally-extending channels formed therein. The wall structure may include an inner tube defining the lumen and an outer jacket that defines at least one channel.

The invention further provides a method of draining unwanted bodily fluids in a patient requiring long-term drainage, the method comprising the step of implanting into a patient in need thereof an implantable shunt system as described above. The catheter is suitably adjacent to a biocompatible matrix, the matrix capable of delivering a therapeutic agent over a prolonged period of time. The fluids suitably comprise cerebrospinal fluid. The fluids may comprise a carrier and at least one therapeutic agent or metabolites thereof.

The invention further provides a method for delivering a therapeutic agent to a patient having hydrocephaly, the method comprising the step of surgically implanting into the patient a cerebrospinal fluid shunt system as described above.

The invention further provides the use of a therapeutic agent in the manufacture of an implantable shunt system as described above. The invention further provides the use of a therapeutic agent in the manufacture of a cerebrospinal fluid shunt system as described above.

Other aspects and advantages of the invention will be apparent from the following detailed description of the invention.

### BRIEF DESCRIPTION OF THE DRAWINGS

The features and advantages of the present invention should become apparent from the following description when taken in conjunction with the accompanying drawings, in which:

FIG. 1 is a schematic view of a shunt system according to the present invention;

FIG. 2 is an enlarged cross-sectional view of a first embodiment of a catheter or shunt according to the present invention;

FIG. 3 is an enlarged cross-sectional view of a second embodiment of a catheter or shunt according to the present invention;

FIG. 4 is an enlarged cross-sectional view of a third embodiment of a catheter or shunt according to the present invention;

FIG. 5 is an enlarged cross-sectional view of a fourth embodiment of a catheter or shunt according to the present invention;

FIG. 6 is a cross-sectional view of the catheter/shunt illustrated in FIG. 5 along line 6-6;

FIG. 7 is a schematic view of a rechargeable catheter/shunt system having a reservoir according to the present invention; and

FIG. 8 is a schematic view of an alternate rechargeable catheter/shunt system having a reservoir according to the present invention.

### DETAILED DESCRIPTION OF THE INVENTION

The present invention relates to a catheter, or shunt, for use in being implanted within a patient as part of a shunt system for draining fluid between different areas of the patient's body. A shunt system for the treatment of hydrocephalus provides one example. However, the implantable catheter/shunt of the present invention can be used in other fluid-draining or like applications.

The implantable catheter/shunt of the present invention is particularly useful in applications where it is desirable to deliver a therapeutic agent, drug, or like useful substance to a location adjacent the inner and/or outer surfaces of the catheter/shunt and/or within the lumen of the catheter/shunt. As an example, the catheter/shunt of the present invention can be used to gradually release one or more therapeutic agents that minimize the risk of blockage or obstruction of the lumen of the catheter/shunt or that minimize the risk of infection. Of course, the catheter/shunt of the present invention can also be used to release other useful substances for other intended purposes.

A typical cerebrospinal shunt system 10 is illustrated in FIG. 1 within patient “P”. The system 10 includes a ventricular catheter 12 extending through a burr hole surgically formed in the skull of the patient. The catheter 12 has a proximal end, or inflow end, 14 positioned in the patient's ventricle and a longitudinally-extending lumen that provides a drainage path for the flow of cerebrospinal fluid to a flow control mechanism, or unidirectional valve, 16. The valve 16 connects to a drainage shunt 18 which provides a flow path 20 to the patient's peritoneal cavity where the drained cerebrospinal fluid can be reabsorbed into the blood through the peritoneum, the membrane which lines the gastro-intestinal organs. Alternatively, the shunt 18 can provide a path 22 (shown in phantom) to the right atrium of the heart directly into blood circulation.

The entire shunt system 10 is positioned, or implanted, under the skin. For example, the catheter 12 and shunt 18 can be tunnelized in the subcutaneous tissue of the patient and can be made of silicone or a like polymer that is well tolerated by the body. The valve 16 is inserted under the skin onto the cranium behind the ear, or alternatively, into the pectoral region or into the flank.

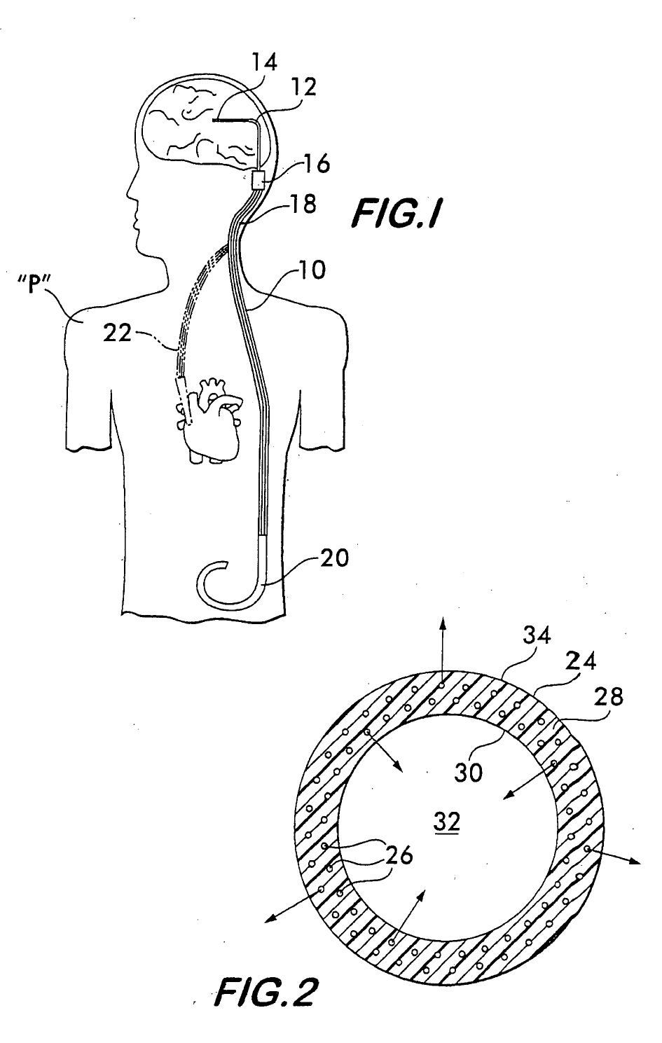

A first embodiment of a catheter/shunt 24 according to the present invention is illustrated in cross-section in FIG. 2. The catheter/shunt 24 can be used as a ventricular catheter, a drainage shunt, or both, or can be used in other drainage or like applications. At least a predetermined length of the catheter/shunt 24 is made of a polymer, such as silicone, that contains therein a therapeutic agent 26. For example, the therapeutic agent 26 can be mixed with the polymer before manufacture of the catheter/shunt so that, upon manufacture of the catheter/shunt, the therapeutic agent 26 is distributed uniformly throughout the formed walls of the catheter/shunt. Accordingly, the wall 28 of the catheter/shunt 24 is impregnated with the therapeutic agent 26, and the therapeutic agent 26 can be slowly released therefrom in situ to deliver a controlled amount of the therapeutic agent 26 within the patient over a predetermined period of time.

By way of example, the therapeutic agent 26 can be rapamycin, an mTOR inhibitor, an antimicrobial, an antibiotic or other active agent or useful substance. As shown by arrows in FIG. 2, the therapeutic agent 26 can be released through an inner peripheral surface 30 of the wall 28 into the lumen 32 of the catheter/shunt 24 and/or through an outer peripheral surface 34 of the wall 28. The gradual diffusion of the therapeutic agent 26, such as rapamycin, through surfaces 30 and 34 can effectively prevent bacterial growth within and around the catheter/shunt 24 and prevent tissue ingrowths that might block or obstruct the drainage of fluid through the lumen 32. As a specific example, the therapeutic agent 26 can be used to prevent undesired choroid plexus attachment to the catheter/shunt 26.

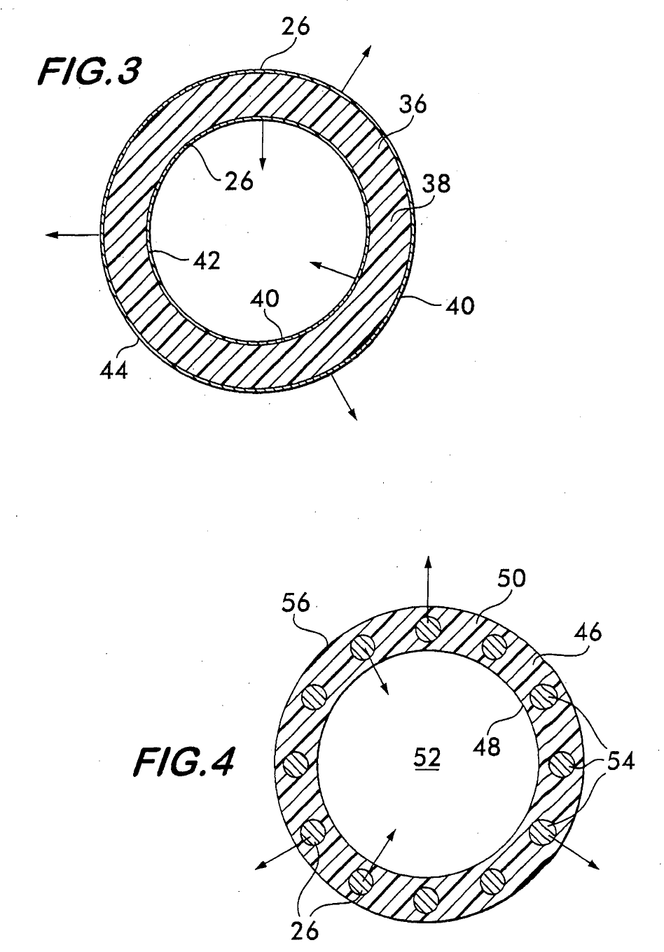

A second embodiment of a catheter/shunt 36 according to the present invention is illustrated in cross-section in FIG. 3. The wall 38 of the catheter/shunt 36 is coated with a film or coating 40 containing a therapeutic agent 26. The film or coating 40 can be made of a solution including a mixture of the therapeutic agent 26 and a polymer carrier solution that is applied to one or both of the inner peripheral surface 42 and outer peripheral surface 44 of the wall 38 by dip-coating, spray-coating, brush-coating, spin-coating or like techniques. When the catheter/shunt 36 is implanted in a patient, the therapeutic agent 26 is gradually released therefrom over a predetermined period of time. The therapeutic agent 26 can be any of those discussed above.

A catheter/shunt 24 shown in FIG. 2 can be applied with the coating or film 40 illustrated in FIG. 3. In this case, the film 40 can be relied upon to provide an initial short-term burst/release of therapeutic agent 26 followed by a slower long-term release of the therapeutic agent 26 impregnated within the wall of the catheter/shunt. Alternatively, the film 40 can contain one type of therapeutic agent while a different type of therapeutic agent is impregnated within the wall. For example, the therapeutic agent in the film 40 can be a compound that prevents infection, and the therapeutic agent impregnated within the wall can prevent tissue ingrowths. Of course, other combinations of useful substances can also be utilized.

A third illustrated embodiment of a catheter/shunt 46 according to the present invention is shown in cross-section in FIG. 4. An advantage of this particular embodiment is that it enables recharging and/or refilling of the therapeutic agent carried by the catheter/shunt thereby extending the period of time in which a therapeutic agent can be delivered from the implanted catheter/shunt. This period of time can include the entire useful life of the implanted catheter/shunt.

The inner peripheral surface 48 of the wall 50 of the catheter/shunt 46 defines a centrally-extending lumen 52, and one or more hollow channels 54 extend longitudinally within the wall 50 between the inner peripheral surface 48 and an outer peripheral surface 56 of the wall 50. The hollow channels 54 are filled with a therapeutic agent 26 that is permitted to gradually migrate through one or both of the inner and outer peripheral surfaces, 48 and 56. The specific embodiment shown in FIG. 4 is preferably an extruded flexible tube in which the lumen 52 and channels 54 are formed during an extrusion tube-forming process. The channels 54 can extend the length of the catheter/shunt or in only a predetermined length thereof.

A substantially ring-shaped end-cap or connector (not shown) can be fitted about an end tip of the catheter/shunt 46 to plug the ends of the channels 54 or to provide U-shaped passages that interconnect the ends of one or more channels 54 and that provide reversely-turned channels. For example, the therapeutic agent may be permitted to flow in a first direction along the length of a first channel and then in a reverse direction in an adjacent interconnected channel. The therapeutic agent 26 can be any discussed above, and the catheter/shunt 46 can contain multiple types of therapeutic agents in different ones of unconnected channels within the wall 50. The wall 50 can also be impregnated with a therapeutic agent in accordance with the embodiment illustrated in FIG. 2 and/or can be provided with films containing a therapeutic agent in accordance with the embodiment illustrated in FIG. 3.

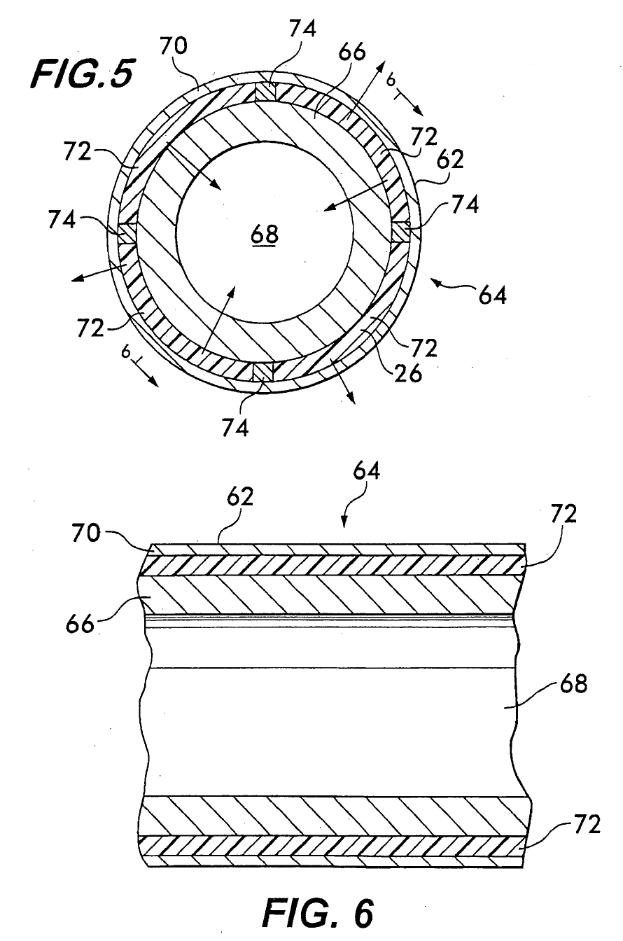

A fourth illustrated embodiment of a catheter/shunt 62 according to the present invention is shown in FIGS. 5 and 6. The catheter/shunt 62 has a wall structure 64 including an inner tube 66 defining a lumen 68 and an outer tube, or jacket, 70 that envelopes the inner tube 66. Spacing is provided between the inner tube 66 and jacket 70 providing one or more channels 72 therebetween for holding a supply of the therapeutic agent 26. One or more stabilizers 74 can be provided to ensure proper spacing between the inner tube 66 and jacket 70. The stabilizers 74 can also be used to define and isolate separate longitudinally-extending channels 72 within the wall structure 64. The stabilizers 74 can be designed to permit cross flow between adjacent channels or prevent cross-flow between adjacent channels. The use of separate channels may be desirable to ensure uniform distribution of the therapeutic agent about the catheter/shunt 62 or may permit different therapeutic agents to be carried separately in the various channels 72.

The supply of therapeutic agent 26 in channels 72 gradually migrates, or diffuses, through one or both of the inner tube 66 and the jacket 70. This provides a slow release of the therapeutic agent over an extended period of time. Ring-shaped caps, connectors or the like (not shown) can be fitted over the end tips of the catheter/shunt 62 to seal the ends of the channels 72. The same or different therapeutic agent can be impregnated within the inner tube 66 and/or jacket 70 and/or be provided in a coating applied to the inner tube 66 and/or jacket 70.

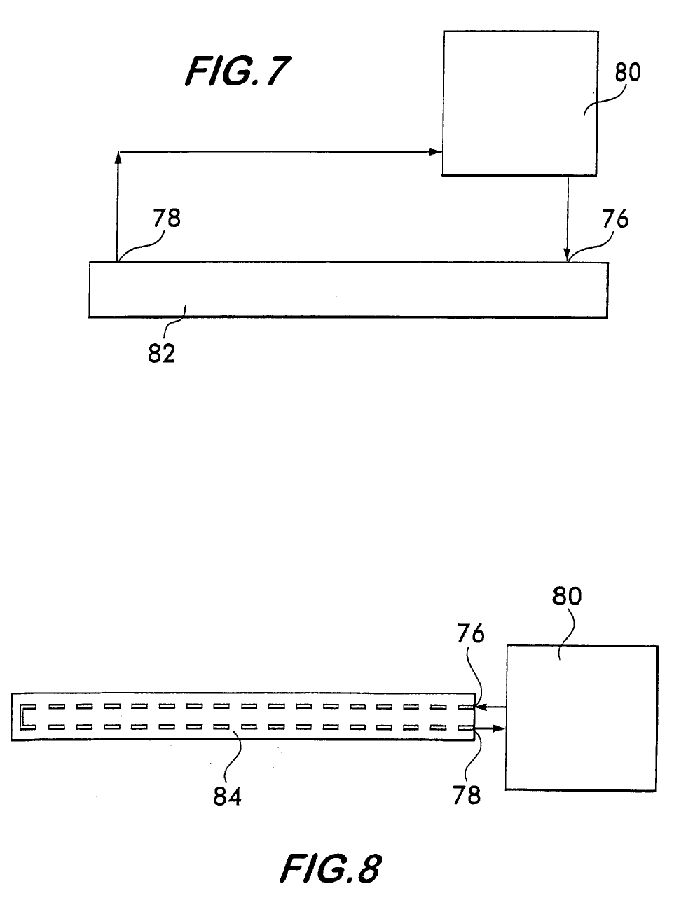

The catheter/shunts 46 and 62 having channels, 54 and 72, permit the supply of therapeutic agent carried thereby to be recharged, flushed, refilled, and/or circulated. This extends the useful life of the implanted shunt system and the period of time over which the therapeutic agent can be delivered in situ to the patient. For this purpose, the channels can be provided with an entry port 76 and an exit port 78 that are each interconnected to a reservoir 80 containing an additional supply of the therapeutic agent. For example, the reservoir 80 can be implanted underneath the skin behind a patient's ear and may contain a transient bolus dose of the therapeutic agent. The reservoir itself may be refillable with the use of a syringe or the like. In addition, an implantable pump (not shown) can be provided to force circulation of the therapeutic agent from the reservoir and into the channels of the catheter/shunt. For example, the pump can be a mechanical pump that is actuated by pressure when pressure is applied to the skin where the pump is implanted. FIG. 7 shows a system in which circulation is directed one-way along a predetermined length of a catheter 82, while FIG. 8 shows a system in which circulation of therapeutic agent is reversed within the catheter/shunt 84 to permit the entry and exit ports to be closely positioned to one another and the reservoir.

In all of the above referenced embodiments of the present invention, the therapeutic agent or agents can be any substance considered useful for delivery in situ within a patient adjacent to or within the lumen of the catheter/shunt. A particularly useful substance contemplated by the present invention is an mTOR inhibitor such as rapamycin. Rapamycin is a macrolide antibiotic which can prevent tissue and bacterial growth and which possesses anti-inflammatory activity. Accordingly, tissue ingrowths into the lumen of the catheter/shunt, lumen blockage or obstruction, and tissue attachment to the catheter/shunt can be prevented by the gradual release of rapamycin therein. Alternatively, the therapeutic agent can be analogs of rapamycin or other mTOR inhibitors. Substances such as drugs, sterilants, plasticizers, antimicrobials, and the like can also be utilized as therapeutic agents.

While preferred shunt systems and catheters/shunts have been described in detail, various modifications, alterations, and changes may be made without departing from the spirit and scope of the present invention as defined in the appended claims.

### Methods of Drawing Fluids

In another aspect, the invention provides for the use of an implantable shunt system for draining unwanted fluids in a patient. The implantable shunt system provides sufficient levels of an mTOR inhibitor to avoid cellular ingrowth in the shunt system. Thus, the shunt system minimizes or eliminates bacterial growth and cellular attachment, e.g., choroid plexus attachment, to the shunt.

As used herein, the term “mTOR inhibitor” means a compound or ligand, or a pharmaceutically acceptable salt thereof, that inhibits cell replication by blocking the progression of the cell cycle from G1 to S. The term includes the neutral tricyclic compound rapamycin (sirolimus) and other rapamycin compounds, including, e.g., rapamycin derivatives, rapamycin analogues, other macrolide compounds that inhibit mTOR activity, and all compounds included within the definition below of the term “a rapamycin”. These include compounds with a structural similarity to “a rapamycin”, e.g., compounds with a similar macrocyclic structure that have been modified to enhance therapeutic benefit. FK-506 can also be used in the method of the invention.

As defined herein, the term “a rapamycin” defines a class of immunosuppressive compounds which contain the following rapamycin nucleus:

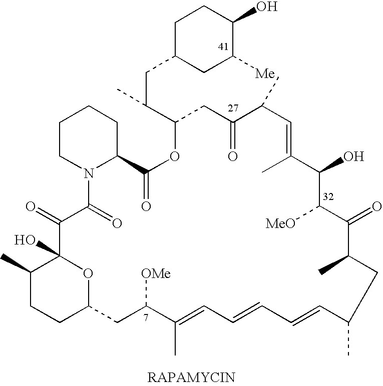

The term “desmethylrapamycin” refers to the class of immunosuppressive compounds which contain the basic rapamycin nucleus shown, but lacking one or more methyl groups. In one embodiment, the rapamycin nucleus is missing a methyl group from either positions 7, 32, or 41, or combinations thereof. The synthesis of other desmethylrapamycins may be genetically engineered so that methyl groups are missing from other positions in the rapamycin nucleus. Production of desmethylrapamycins has been described. See, e.g., 3-desmethylrapamycin [U.S. Pat. No. 6,358,969], and 17-desmethylrapamycin [U.S. Pat. No. 6,670,168].

The terms “desmethylrapamycin” and “—O-desmethylrapamycin” are used interchangeably throughout the literature and the present specification, unless otherwise specified.

The rapamycins used according to this invention include compounds which may be chemically or biologically modified as derivatives of the rapamycin nucleus, while still retaining immunosuppressive properties. Accordingly, the term “a rapamycin” includes esters, ethers, oximes, hydrazones, and hydroxylamines of rapamycin, as well as rapamycins in which functional groups on the nucleus have been modified, for example through reduction or oxidation. The term “a rapamycin” also includes pharmaceutically acceptable salts of rapamycins, which are capable of forming such salts, either by virtue of containing an acidic or basic moiety.

As used herein, pharmaceutically acceptable salts include, but are not limited to, hydrochloric, hydrobromic, hydroiodic, hydrofluoric, sulfuric, citric, maleic, acetic, lactic, nicotinic, succinic, oxalic, phosphoric, malonic, salicylic, phenylacetic, stearic, pyridine, ammonium, piperazine, diethylamine, nicotinamide, formic, urea, sodium, potassium, calcium, magnesium, zinc, lithium, cinnamic, methylamino, methanesulfonic, picric, tartaric, triethylamino, dimethylamino, and tris(hydroxymethyl)aminomethane. Additional pharmaceutically acceptable salts are known to those skilled in the art.

In one embodiment, the esters and ethers of rapamycin are of the hydroxyl groups at the 42- and/or 31-positions of the rapamycin nucleus, esters and ethers of a hydroxyl group at the 27-position (following chemical reduction of the 27-ketone), and that the oximes, hydrazones, and hydroxylamines are of a ketone at the 42-position position (following oxidation of the 42-hydroxyl group) and of 27-ketone of the rapamycin nucleus.

In another embodiment, 42- and/or 31-esters and ethers of rapamycin are described in the following patents: alkyl esters (U.S. Pat. No. 4,316,885); aminoalkyl esters (U.S. Pat. No. 4,650,803); fluorinated esters (U.S. Pat. No. 5,100,883); amide esters (U.S. Pat. No. 5,118,677); carbamate esters (U.S. Pat. No. 5,118,678); silyl ethers (U.S. Pat. No. 5,120,842); aminoesters (U.S. Pat. No. 5,130,307); acetals (U.S. Pat. No. 5,51,413); aminodiesters (U.S. Pat. No. 5,162,333); sulfonate and sulfate esters (U.S. Pat. No. 5,177,203); esters (U.S. Pat. No. 5,221,670); alkoxyesters (U.S. Pat. No. 5,233,036); O-aryl, -alkyl, -alkenyl, and-alkynyl ethers (U.S. Pat. No. 5,258,389); carbonate esters (U.S. Pat. No. 5,260,300); arylcarbonyl and alkoxycarbonyl carbamates (U.S. Pat. No. 5,262,423); carbamates (U.S. Pat. No. 5,302,584); hydroxyesters (U.S. Pat. No. 5,362,718); hindered esters (U.S. Pat. No. 5,385,908); heterocyclic esters (U.S. Pat. No. 5,385,909); gem-disubstituted esters (U.S. Pat. No. 5,385,910); amino alkanoic esters (U.S. Pat. No. 5,389,639); phosphorylcarbamate esters (U.S. Pat. No. 5,391,730); carbamate esters (U.S. Pat. No. 5,411,967); carbamate esters (U.S. Pat. No. 5,434,260); amidino carbamate esters (U.S. Pat. No. 5,463,048); carbamate esters (U.S. Pat. No. 5,480,988); carbamate esters (U.S. Pat. No. 5,480,989); carbamate esters (U.S. Pat. No. 5,489,680); hindered N-oxide esters (U.S. Pat. No. 5,491,231); biotin esters (U.S. Pat. No. 5,504,091); O-alkyl ethers (U.S. Pat. No. 5,665,772); and PEG esters of rapamycin (U.S. Pat. No. 5,780,462). The preparation of these esters and ethers is described in the patents listed above.

In yet another embodiment, 27-esters and ethers of rapamycin are described in U.S. Pat. No. 5,256,790. The preparation of these esters and ethers is described in the patent listed above.

In still another embodiment, oximes, hydrazones, and hydroxylamines of rapamycin are described in U.S. Pat. Nos. 5,373,014, 5,378,836, 5,023,264, and 5,563,145. The preparation of these oximes, hydrazones, and hydroxylamines is described in the above-listed patents. The preparation of 42-oxorapamycin is described in U.S. Pat. No. 5,023,263.

In another embodiment, rapamycins include rapamycin [U.S. Pat. No. 3,929,992], rapamycin 42-ester with 3-hydroxy-2-(hydroxymethyl)-2-methylpropionic acid [U.S. Pat. No. 5,362,718], and 42-O-(2-hydroxy)ethyl rapamycin [U.S. Pat. No. 5,665,772]. The preparation and use of hydroxyesters of rapamycin, including CCI-779, is described in U.S. Pat. Nos. 5,362,718 and 6,277,983.

As used herein, the term “a CCI-779” means rapamycin 42-ester with 3-hydroxy-2-(hydroxymethyl)-2-methylpropionic acid (temsirolimus), and encompasses prodrugs, derivatives, pharmaceutically acceptable salts, or analogs thereof.

Examples of a rapamycin include, e.g., rapamycin, 32-deoxorapamycin, 16-pent-2-ynyloxy-32-deoxorapamycin, 16-pent-2-ylyloxy-32(S)-dihydro-rapamycin, 16-pent-2-ylyloxy-32(S)-dihydr-o-40-O-(2-hydroxyethyl)-rapamycin, 40-O-(2-hydoxyethyl)-rapamycin, rapamycin 42-ester with 3-hydroxy-2-(hydroxymethyl)-2-methylpropionic acid (CCI-779), 40-[3-hydroxy-2-(hydroxymethyl)-2-meth-ylpropanoate]-rapamycin, or a pharmaceutically acceptable salt thereof, as disclosed in U.S. Pat. No. 5,362,718, ABT578, or 40-(tetrazolyl)-rapamycin, 40-epi-(tetrazolyl)-rapamycin, e.g., as disclosed in International Patent Publication No. WO 99/15530, or rapamycin analogs as disclosed in International Patent Publication No. WO 98/02441 and WO 01/14387, e.g., AP23573. In another embodiment, the compound is Certican™ (everolimus, 2-O-(2-hydroxy)ethyl rapamycin, Novartis, U.S. Pat. No. 5,665,772).

In one embodiment, one or more of the components of the shunt system, e.g., the catheter, the pump, etc., is inserted in immediate proximity to a biocompatible matrix capable of delivering a therapeutic agent over a prolonged period of time.

Thus, the invention provides a method of draining unwanted fluids in a patient using a shunt system which itself supplies or is in close proximity to a matrix providing an extended release or rechargeable source of an mTOR inhibitor in sufficient amounts to prevent cellular growth in or tissue attachment to one or more components of the shunt system. A biocompatible, biodegradable resorbable matrix material such as collagen, fibrin or chitosan, may be used. Alternatively, a suitable biocompatible, nonbiodegradable matrix may be also be used. Suitable biocompatible matrices have been described in the literature. Many such matrices may be obtained commercially, e.g., from Advanced Nanotechnology, Atrigel® drug delivery system [QLT USA], and loaded with the desired compound using manufacturer's methods. Alternatively, the mTOR inhibitor is delivered by the shunt system itself, e.g., by impregnation in a component of the shunt system, or via bolus dose.

The mTOR inhibitor may be provided in amounts which are within the range considered therapeutic for certain indications, e.g., in the range of about 5 to about 175 mg, or about 5, about 10, about 20, or to about 25 mg. However, because the invention provides for the mTOR inhibitor to be provided locally, and in view of the fact that it is desirable to minimize the amount of fluid delivered, the mTOR inhibitor can be provided in lower amounts which are still sufficient to inhibit cellular growth in one or more of the components of the shunt system and, particularly, in the drainage catheter. For example, suitable amounts may range from about 0.0001 mg to 1 mg, which is released daily, weekly, or as otherwise provided by the extended release system.

Typically, the source of the unwanted fluids to be drained will vary, depending upon the application for which the shunt is utilized. For example, in a hydrocephaly patient, the shunt will drain cerebrospinal fluid. In another example, where the shunt system is used for a glaucoma patient, the shunt may drain intravitreal fluid.

In addition, where the shunt system is used to deliver a bolus or other dose of a therapeutically effective amount of a compound, the fluids may include carriers, metabolites of an active compound, and other inactive components of a pharmaceutical composition.

Examples of therapeutic compounds which may be delivered via the shunt system, or drained through the shunt system, include, without limitation, a therapeutically effective amount of an mTOR inhibitor, an antibiotic, drugs useful for treatment of conditions associated with Alzheimer's Disease and other disorders for which delivery to the brain is desirable, drugs useful for the treatment of eye disorders including glaucoma, macular degeneration and the like.

In one embodiment, the invention provides a method for delivering a therapeutic agent to a patient having hydrocephaly, said method comprising the step of surgically implanting into said patient a cerebrospinal fluid shunt system of the invention.

All patents, patent publications, articles, and other documents referenced herein are incorporated by reference. It will be clear to one of skill in the art that modifications can be made to the specific embodiments described herein without departing from the scope of the invention.

1. An implantable shunt system for draining fluid from a body cavity, comprising a catheter and at least one therapeutic agent carried by a wall structure of said catheter, said wall structure having an outer peripheral surface and an inner peripheral surface defining a lumen through which fluid is drained from the body cavity, and said wall structure enabling gradual release of said therapeutic agent from said wall structure in situ after implantation of said catheter body.

2. The implantable shunt system according to claim 1, further comprising a means for recharging or refilling the therapeutic agent carried by said wall structure in situ within a patient.

3. The implantable shunt system according to claim 2, further comprising at least one channel extending within said wall structure between said inner and outer peripheral surfaces, said channel containing a supply of said therapeutic agent which is gradually releasable therefrom through at least one of said inner and outer peripheral surfaces.

4. The implantable shunt system according to claim 3, wherein said at least one channel includes an inlet, and wherein said catheter further comprises a reservoir that carries a supply of said therapeutic agent, that is located external of said wall structure and that is in fluid communication with said inlet.

5. The implantable shunt system according to claim 4, further comprising a pump that can be actuated to pump said therapeutic agent from said reservoir into said channel via said inlet.

6. The implantable shunt system according to claim 5, wherein said at least one channel comprises a plurality of adjacent channels extending in a substantially longitudinal direction within said wall structure along a predetermined length of said catheter body.

7. The implantable shunt system according to claim 6, wherein at least selected ones of said channels interconnect at ends thereof so that said therapeutic agent travels in a first direction along a length of one of said channels and in a reverse direction within an adjacent channel.

8. The implantable shunt system according to claim 6, wherein said reservoir includes multiple separate reservoirs each communicating with a different channel and carrying a supply of a different therapeutic agent.

9. The implantable shunt system according to claim 1, wherein said therapeutic agent is an mTOR inhibitor.

10. The implantable shunt system according to claim 9, wherein said therapeutic agent is a rapamycin.

11. The implantable shunt system according to claim 10, wherein said therapeutic agent is selected from the group consisting of rapamycin and CCI-779.

12. The implantable shunt system according to claim 1, wherein multiple different therapeutic agents are carried by said wall structure.

13. The implantable shunt system according to claim 1, wherein said therapeutic agent is provided in a film formed on a surface of said wall structure.

14. The implantable shunt system according to claim 1, wherein said therapeutic agent is impregnated within a material from which said wall structure is formed and is diffusible through said wall structure.

15. The implantable shunt system according to claim 1, wherein said wall structure of is a single-walled tube extruded with hollow channels extending within the single-wall of the tube.

16. The implantable shunt system according to claim 1, wherein said wall structure includes an inner tube defining said lumen through which fluid is drained from the body cavity and an outer tube or jacket supported a spaced distance about said inner tube defining a chamber therebetween for containing a supply of said therapeutic agent.

17. The implantable shunt system according to claim 14, wherein said wall structure includes at least one stabilizer positioned within said chamber to maintain proper spacing between said inner tube and outer tube or jacket or to divide said chamber into multiple separate chambers.

18. A cerebrospinal fluid shunt system having a ventricular catheter and drainage shunt interconnected directly or indirectly by a flow control mechanism, one of said ventricular catheter and drainage shunt comprising an elongate body defining a lumen therein for passage of cerebrospinal fluid to or from said flow control mechanism, said body formed by a wall structure carrying at least one therapeutic agent therein or thereon, said wall structure releasing said therapeutic agent in situ after implantation of said shunt system.

19. The cerebrospinal fluid shunt system according to claim 18, further comprising means for recharging or refilling the therapeutic agent carried by said wall structure in situ within a patient.

20. The cerebrospinal fluid shunt system according to claim 18, wherein said therapeutic agent is an mTOR inhibitor.

21. The cerebrospinal fluid shunt system according to claim 20, wherein said mTOR inhibitor is a rapamycin.

22. The cerobrospinal fluid shunt system according to claim 21, wherein the rapamycin is selected from the rapamycin and CCI-779.

23. The cerebrospinal fluid shunt system according to claim 18, wherein said therapeutic agent is provided in a film formed on a surface of said wall structure or is impregnated within a material from which said wall structure is composed and is diffusible through said wall structure.

24. The cerebrospinal fluid shunt system according to claim 18, wherein said wall structure provides an outer peripheral surface of said body and an inner peripheral surface of said body, wherein said inner peripheral surface defines said lumen, and wherein at least one channel extends within said wall structure between said inner and outer peripheral surfaces, said channel containing a supply of said therapeutic agent which is slowly releasable therefrom through at least one of said inner and outer peripheral surfaces.

25. The cerebrospinal fluid shunt system according to claim 24, wherein said at least one channel includes an inlet and an outlet permitting flushing, refilling, recharging or circulating of said supply of therapeutic agent in said channel.

26. The cerebrospinal fluid shunt system according to claim 18, further comprising at least one implantable reservoir carrying a supply of said therapeutic agent, being located external of said wall structure, and being in fluid communication with said inlet and outlet.

27. The cerebrospinal fluid shunt system according to claim 26, further comprising an implantable pump located external of said wall structure and adapted to pump said therapeutic agent from said reservoir into said channel.

28. The cerebrospinal fluid shunt system according to claim 27, wherein said wall structure is an extruded flexible single-walled tube having an array of separate longitudinally-extending channels formed therein.

29. The cerebrospinal fluid shunt system according to claim 27, wherein said wall structure includes an inner tube defining said lumen and an outer jacket that defines said at least one channel therebetween.

30. A method of draining unwanted bodily fluids in a patient requiring long-term drainage, the method comprising the step of implanting into a patient in need thereof an implantable shunt system according to claim 1.

31. The method according to claim 30, wherein the catheter is adjacent to a biocompatible matrix, said matrix capable of delivering a therapeutic agent over a prolonged period of time.

32. The method according to claim 30, wherein the fluids comprise cerebrospinal fluid.

33. The method according to claim 30, wherein the fluids comprise a carrier and at least one therapeutic agent or metabolites thereof.

34. The method according to claim 33, wherein the therapeutic agent is an mTOR inhibitor.

35. The method according to claim 33, wherein the therapeutic agent is an antibiotic.

36. A method for delivering a therapeutic agent to a patient having hydrocephaly, said method comprising the step of surgically implanting into said patient a cerebrospinal fluid shunt system according to claim 18.
